(19) United States
(12) Patent Application Publication
(10) Pub. No.: US 2005/0143361 A1
(43) Pub. Date: Jun. 30, 2005

(54) Compositions for pulmonary administration

(76) Inventors: Pertti Happonen, Kuopio (FI); Elina Serkkola, Helsinki (FI); Erkki Nissinen, Espoo (FI)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/502,087

(22) PCT Filed: Jan. 22, 2003
(86) PCT No.: PCT/FI03/00054

(30) Foreign Application Priority Data
Jan. 23, 2002 (FI) 20020126

(51) Int. Cl. A61K 31/573; A61L 9/04; A61K 9/14

(52) U.S. Cl. 514/171; 424/46

(57) ABSTRACT

An inhalation medicament comprising orazipone and a beta2-adrenoreceptor agonist and/or a corticosteroid as a combined preparation. Optionally the medicament also comprises one or more pharmaceutically acceptable additive, diluents or carriers. The beta-2adrenoreceptor agonist and the corticosteroid may comprise, respectively, formoterol and budesonide.

### FIELD OF THE INVENTION

The present invention relates to pharmaceutical compositions useful in the treatment of asthma and other respiratory disorders. More particularly, it relates to compositions comprising anti-inflammatory agent orazipone in combination with a β₂-adrenoreceptor agonist and/or a corticosteroid.

### BACKGROUND OF THE INVENTION

Asthma is currently treated with drugs that can be classified into two classes, namely anti-inflammatory agents and bronchodilators. Anti-inflammatory drugs such as corticosteroids and sodium cromoglycate do not relieve asthma symptoms once they occur, but rather they control the underlying inflammation. One of the drawbacks of anti-inflammatory drugs is that their onset of action is relatively slow. Therefore, patients often do not recognize any immediate therapeutic effects and tend to stop the medication. The acute asthma symptoms can be relieved by bronchodilators such as β₂-adrenoreceptor agonists and theophylline. The short-acting inhaled β₂-adrenoreceptor agonists, e.g. salbutamol and terbutaline, are important for an immediate symptomatic asthma relief, while long-acting β₂-adrenoreceptor agonists, e.g. salmeterol, formoterol and procaterol, are important for treatment of moderate and severe asthma. However, there are currently still various debates on the safety of a regular use of β₂-adrenoreceptor agonists as well as efficiency of long-acting β₂-adrenoreceptor agonists.

Inhalation has become the primary route of administration in the treatment of asthma and other respiratory diseases. This is because, besides providing direct access to the lungs, medication delivered through the respiratory tract provides rapid and predictable onset of action and requires lower dosages compared to the oral route. Typical delivery systems for inhalable drugs are the pressurized metered-dose inhaler (pMDI) comprising a suspension of fine drug particles in a propellant gas; the dry powder inhaler (DPI) comprising fine drug particles as dry powder typically admixed with coarser carrier or diluent such as lactose, and nebulizer comprising drug in aqueous solution or suspension. Inhalable combinations of an anti-inflammatory agent and a bronchodilator have been described e.g. in patent publications EP 416950, EP 416951, WO 93/11773 and WO 98/15280.

Despite recent advances in the understanding and treatment of asthma, there are still problems related to dosing regimens and systemic effects of the anti-asthma drugs. Therefore improvements in the treatment of asthma and other respiratory disorders are desired.

### SUMMARY OF THE INVENTION

It has been found that an inhalation medicament comprising, as a combined preparation, orazipone and a β₂-adrenoreceptor agonist and/or a corticosteroid, provides improved disease control of asthma and other respiratory disorders.

Accordingly, the present invention provides an inhalation medicament comprising orazipone and a β₂-adrenoreceptor agonist and/or a corticosteroid as a combined preparation.

The present invention also provides a method for treating asthma and other respiratory disorders which comprises the simultaneous, sequential or separate administration of an effective amount of orazipone and a β₂-adrenoreceptor agonist and/or a corticosteroid.

The present invention also provides an inhaler device containing orazipone and β₂-adrenoreceptor agonist and/or a corticosteroid as therapeutically active ingredients for pulmonary administration.

The active ingredients of the combination are preferably presented, separately or in admixture, as a pharmaceutical formulation together with one or more pharmaceutically acceptable additive, diluent or carrier.

The active ingredients are preferably provided as micronized particles, e.g. having mass median diameter of less than 10 μm. Preferably, the medicament is provided in the form of dry inhalation powder comprising the active ingredients in admixture with carrier particles.

### BRIEF DESCRIPTION OF THE DRAWINGS

FIG. 1 shows the effect of budesonide and orazipone and combination thereof on LPS-induced IL-8 production in peripheral blood mononuclear cells.

### DETAILED DESCRIPTION OF THE INVENTION

Orazipone or 3-[(4-methylsulfonylphenyl)-methylene]-2,4-pentanedione is a locally acting anti-inflammatory agent that decomposes in the blood circulation. A method for preparing orazipone for the treatment of inflammatory bowel disease is described in European Patent No. 440324 B1. A powdered inhalation composition of orazipone for the treatment of asthma is disclosed in U.S. Pat. No. 6,201,027.

Suitable β₂-adrenoreceptor agonists to be combined with orazipone include salbutamol, formoterol, fenoterol, procaterol, salmeterol, clenbuterol, bambuterol, bitolterol, carbuterol, hexoprenaline, ibuterol, pirbuterol, reproterol, sulfonterol, tulobuterol, clorprenaline, etafedrine, isoetharine, isoproterenol, mabuterol, metaproterenol, methoxyphenamine, terbutaline and the like and their salts, esters, solvates, isomers including enantiomers and diastereomers. The preferred β₂-adrenoreceptor agonist is formoterol or a pharmaceutically acceptable salt, hydrate or isomer thereof. Preferred salt is formoterol fumarate, particularly in the form of dihydrate. Other suitable salts include acid addition salts of inorganic and organic acids, e.g. chloride, sulphate, tartrate, citrate, lactate and succinate salts or solvates thereof.

Suitable corticosteroids to be combined with orazipone include beclomethasone, budesonide, fluticasone, mometasone, betamethasone, triamcinolone, triamcinolone acetonide, flunisonide, ciclesonide, rofleponide, dexamethasone and the like and their salts and esters and solvates. The preferred corticosteroid is budesonide.

One preferred inhalation medicament according to the invention comprises a combination of orazipone, a β₂-adrenoreceptor agonist and a corticosteroid. Another preferred inhalation medicament according to the invention comprises a combination of orazipone and a corticosteroid. One particularly preferred inhalation medicament according to the invention comprises orazipone, formoterol or a salt or hydrate thereof and budesonide as a combined preparation. Another particularly preferred inhalation medicament according to the invention comprises orazipone and budesonide as a combined preparation.

The combination of the invention is particularly useful in the treatment of asthma and other respiratory diseases, such as mild, moderate and severe asthma, allergic and non-allergic asthma, acute condition of asthma, intermittent asthma, episodes in chronic asthma, chronic obstructive pulmonary disease (COPD) and adult respiratory distress syndrome (ARDS). The treatment may be symptomatic or prophylactic treatment.

The active ingredients of the combination are presented, separately or together, as a pharmaceutical formulation, optionally together with one or more pharmaceutically acceptable additive, diluent or carrier. For example, the combined preparation may contain the active ingredients in admixture, optionally together with one or more pharmaceutically acceptable additive, diluent or carrier. Alternatively, one or more of the active ingredients may be presented in a separate pharmaceutical formulation such separate formulations being packaged as a combined preparation, optionally together a package insert instructing the patient to the correct use of the medicament.

The active ingredients are preferably in the form of micronized particles, preferably having mass median particle diameter of less than about 10 μm, suitably from about 1 to about 5 μm.

The amount of orazipone and β₂-adrenoreceptor agonist and/or corticosteroid to be included in the composition is selected such as to achieve the desired therapeutical effect.

Suitable daily dose of orazipone is from about 100 μg to about 5000 μg, preferably from about 500 to about 2000 μg, depending on the age and weight of the patient and the severity and type of the disease.

Suitable daily dose of β2-adrenoreceptor agonist and corticosteroid vary with the particular compound, but daily amounts which are used in monotherapy are usually suitable. For example, formoterol fumarate is generally administered at a dose of from about 10 to about 150 μg daily, typically 12 or 24 μg twice daily. Budesonide is generally administered at a dose of from about 200 to 1600 μg daily. The suitable amounts depend on the age and weight of the patient and the severity and type of the disease.

Preferably the medicament of the invention is in the form of a dry inhalation powder composition. Such compositions may be prepared e.g. by agglomeration of the micronized particles of the active ingredients and possibly the micronized carrier particles using methods known in the art.

It is particularly preferred that the dry inhalation powder composition is a mixture of the micronized particles of the active ingredients and carrier particles, the carrier particles being typically of coarser particle size. A method of preparing such mixtures typically comprises adding the micronized active ingredients and part of the carrier particles into a blender and mixing until the powder mixture is homogenous. The mixture is then sieved to reduce the number of particle clusters present. Thereafter the rest of carrier particles is added and mixed until the powder is again homogenous.

Particularly preferred carrier materials in dry inhalation powder compositions are carbohydrates. Carbohydrates suitable for use as a dry powder carrier material include, for example, monosaccharides such as fructose, maltose or glucose; disaccharides such as lactose sucrose or trehalose; polysaccharides such as raffinose or melezitose; and alditols such as mannitol, xylitol, lactitol and the like. Preferred carrier is lactose or glucose, lactose being most preferred.

If the medicament contains a carrier, e.g. lactose, the total amount of the active ingredients is about 0.05-50% (w/w), preferably about 1-10% (w/w), based on total weight of the composition.

The mass median particle diameter of the carrier is preferably between 5 and 150 μm, more preferably between 10 and 100 μm, most preferably between 15 and 80 μm.

The medicament may alternatively be in the form of a pressurized aerosol where fine drug particles are suspended in a propellant gas. Examples of aerosol carriers include non-chlorofluorocarbon-based carriers such as HFA (hydrofluoroalkane). Pressurized aerosols can be prepared according to the methods well known in the art.

The medicament of the invention may also comprise additives such as solubilizers, stabilizers, flavouring agents, colorizing agents and preserving agents.

For administration by inhalation, the medicament according to the invention is conveniently delivered by conventional means. For example, the medicament can be delivered from inhaler devices well known in the art such as pressurized metered dose inhalers, dry powder inhalers or nebulizers. When the medicament is in the form of dry inhalation powder, it can be filled in e.g. capsules, cartridges, blister packs or a reservoir, from which the powder may be administered by means of a dry powder inhaler.

In case, one or more of the active ingredients are in separate pharmaceutical formulations, the separate formulations may be filled e.g. in a multi-reservoir type inhaler as described e.g. in WO 00/64519. Alternatively, the formulations may be filled in separate inhalers packaged as a combined preparation.

The medicament according to the invention may be administered to a patient daily or periodically, e.g. one month on treatment and one month off treatment. The medicament may be administered as divided doses from 1 to 4 doses a day.

Suitably, the compositions of the invention comprise the active ingredients in amounts such that each actuation provides therapeutically effective dose. For example, the medicament suitably contains, per dose, from 6 to 50 μg, particularly from 12 to 24 μg, of formoterol fumarate dihydrate, from 50 to 600 μg, particularly from 100 to 400 μg, of budesonide and from 100 to 5000 μg, particularly from 200 to 2000 μg, of orazipone.

Thus, the medicament may contain, per dose, 12 μg of formoterol fumarate dihydrate, 200 μg of budesonide and 1000 μg of orazipone. Administration of one to two such doses by inhalation twice daily would be effective in most cases of moderate persistent asthma and is likely to suffice in many severe asthmatics, too.

An example of a particularly preferred embodiment of the invention is an inhalation medicament in the form of dry inhalation powder comprising
a) formoterol or a pharmaceutically acceptable salt thereof having mass median particle diameter of less than about 10 μm, preferably from about 1 to about 5 μm;
b) budesonide having mass median particle diameter of less than about 10 μm, preferably from about 1 to about 5 μm;
c) orazipone having mass median particle diameter of less than about 10 μm, preferably from about 1 to about 5 μm; and optionally
d) carrier having mass median particle diameter between 5 and 150 μm, preferably between 10 and 100 μm, more preferably between 15 and 80 μm.

In the above embodiment, the amount of formoterol or a pharmaceutically acceptable salt thereof is preferably 0.01-5%, more preferably 0.05-1%, by weight of the composition; the amount of budesonide is preferably 0.1-50%, more preferably 0.5-10%, by weight of the composition; and the amount of orazipone is preferably 0.5-50%, more preferably 1-20%, by weight of the composition and the amount of the carrier is preferably 50-99.9%, more preferably 90-99.5%, by weight of the composition.

An example of another particularly preferred embodiment of the invention is an inhalation medicament in the form of dry inhalation powder comprising
a) budesonide having mass median particle diameter of less than about 10 μm, preferably from about 1 to about 5 μm;
c) orazipone having mass median particle diameter of less than about 10 μm, preferably from about 1 to about 5 μm; and optionally
d) carrier having mass median particle diameter between 5 and 150 μm, preferably between 10 and 100 μm, more preferably between 15 and 80 μm.

The invention is further illustrated by the following examples and experiments, which are not meant to limit the scope of the invention.

### EXAMPLE 1

### Dry Inhalation Powder (Per Dose)

| | |
|---|---|
| Formoterol fumarate dihydrate (micronized) | 12 μg |
| Budesonide (micronized) | 200 μg |
| Orazipone (micronized) | 1000 μg |
| Lactose monohydrate Ph. Eur. | 8 mg |

### EXAMPLE 2

### Dry Inhalation Powder (Per Dose)

| | |
|---|---|
| Budesonide (micronized) | 200 μg |
| Orazipone (micronized) | 1000 μg |
| Lactose monohydrate Ph. Eur. | 8 mg |

Micronized active ingredients and part of the lactose were added into a blender. The powder mixture was mixed until it was homogenous. The mixture was then sieved to reduce the number of particle clusters present. Thereafter the rest of lactose was added and the powder was again mixed until it was homogenous. Powder was poured into the supply chamber of the multi-dose powder inhaler Easyhaler (Orion Corporation trademark) for a supply of 200 doses.

### EXPERIMENTS

Methods

Peripheral Blood Mononuclear Cells (PBMC)

Leukocyte-rich buffy coats were obtained from the Finnish Red Cross Blood Transfusion Service (Helsinki, Finland) and mononuclear cells were isolated by centrifugation on Ficoll-Paque (Amersham Pharmacia Biotech, Uppsala, Sweden). After being washed, the cells were resuspended in RPMI-1640 medium containing 25 mM Hepes (Life Technologies, Paisley, UK) 10% heat-inactivated human AB serum (The Finnish Red Cross Blood Transfusion Service, Helsinki, Finland), 2 mM L-Glutamine (Sigma Chemical, St Louis, Mo., USA) and antibiotics: 50U penicillin-50 μg/ml (Sigma Chemical). Resuspended cells were stored at 4° C. overnight.

Test Compounds

Orazipone and Budesonide (Sigma Chemical, St Louis, Mo., USA) were dissolved in DMSO and diluted prior to use in RPMI 1640. The final DMSO concentration was 0.05%.

Measurement of Cytokine Production

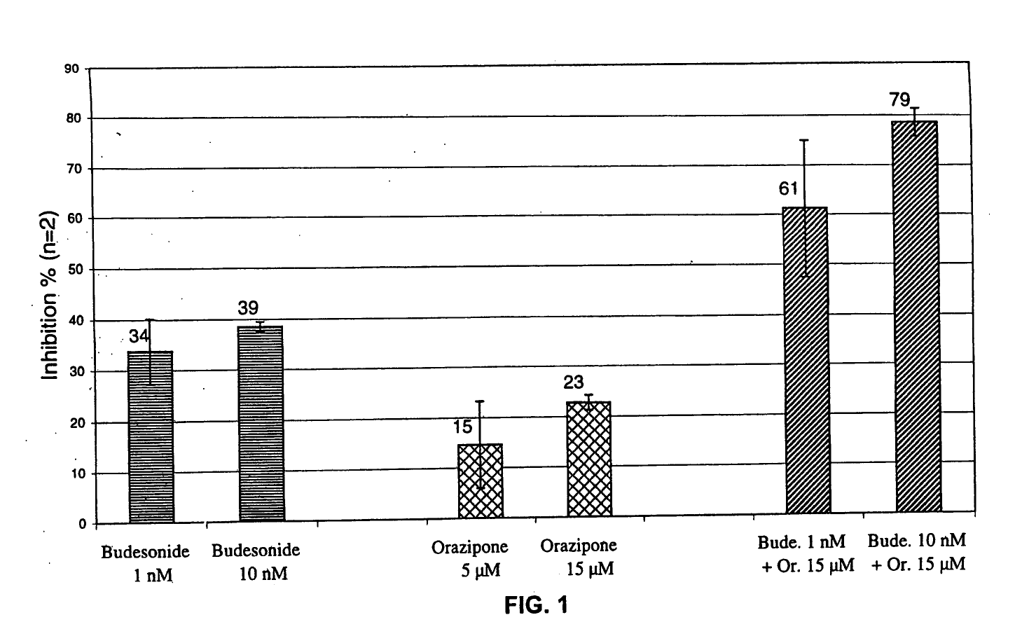

On day two, the cells were sentrifuged and resuspended in fresh RPMI-1640 medium. Cells were plated into 24 well culture plates in one ml RPMI-1640 at a concentration of 1×10⁶ cells/ml. Budesonide were added 30 min before the cells were stimulated with LPS (50 ng/ml) E. Coli (026:B6) (Sigma Chemical, St Louis, Mo., USA). Orazipone was added 2 h after LPS stimulation. After 24 h stimulation, supernatants were collected and frozen at −75° C. until assayed for IL-8 using human ELISA kits from R&D Systems (Abingdon, UK). The cytokine levels were determined according to manufacturer's instructions. The effect of budesonide and orazipone and combination thereof on LPS-induced IL-8 production in peripheral blood mononuclear cells is summarized in FIG. 1. The combination shows a superior and synergistic effect in inhibiting IL-8 production as compared to the effect of budesonide or orazipone alone. The terms “Or.” and “Bude.” denote orazipone and budesonide, respectively.

1. An inhalation medicament comprising orazipone and a β₂-adrenoreceptor agonist and/or a corticosteroid as a combined preparation.

2. An inhalation medicament according to claim 1, wherein the β₂-adrenoreceptor agonist is salbutamol, formoterol, fenoterol, procaterol, salmeterol, clenbuterol, bambuterol, bitolterol, carbuterol, hexoprenaline, ibuterol, pirbuterol, reproterol, sulfonterol, tulobuterol, clorprenaline, etafedrine, isoetharine, isoproterenol, mabuterol, metaproterenol, methoxyphenamine, terbutaline, or a salt, ester, solvate or isomer including enantiomers and diastereomers thereof.

3. An inhalation medicament according to claim 2, wherein the β₂-adrenoreceptor agonist is formoterol or a pharmaceutically acceptable salt or ester or hydrate thereof.

4. An inhalation medicament according to claim 1, wherein the corticosteroid is beclomethasone, budesonide, fluticasone, mometasone, betamethasone, triamcinolone, triamcinolone acetonide, flunisonide, ciclesonide, rofleponide, dexamethasone, or a salt, ester or solvate thereof.

5. An inhalation medicament according to claim 4, wherein the corticosteroid is budesonide.

6. An inhalation medicament comprising orazipone and a corticosteroid as a combined preparation.

7. An inhalation medicament according to claim 6, wherein the corticosteroid is beclomethasone, budesonide, fluticasone, mometasone, betamethasone, triamcinolone, triamcinolone acetonide, flunisonide, ciclesonide, rofleponide, dexamethasone, or a salt, ester or solvate thereof.

8. An inhalation medicament according to claim 7, wherein the corticosteroid is budesonide.

9. An inhalation medicament according to claim 1, wherein the active ingredients are in the form of micronized particles having mass median diameter of less than 10 μm.

10. An inhalation medicament according to claim 9, in the form of dry inhalation powder.

11. An inhalation medicament according to claim 10, wherein the active ingredients are in admixture with a carrier.

12. An inhalation medicament according to claim 11, wherein the carrier is lactose.

13. An inhaler device containing orazipone and a β₂-adrenoreceptor agonist and/or a corticosteroid as therapeutically active ingredients for pulmonary administration.

14. An inhaler device according to claim 13, which is a dry powder inhaler.

15. A method for treating asthma and/or other respiratory disorders which comprises the simultaneous, sequential or separate administration to a patient in need of the treatment of an effective amount of orazipone and a β₂-adrenoreceptor agonist and/or a corticosteroid.
